(19) United States
(12) Patent Application Publication
(10) Pub. No.: US 2020/0386772 A1
(43) Pub. Date: Dec. 10, 2020

(54) Method For Detecting Bodily Adhesions By Identifying Markers In Circulating Cells

(71) Applicant: Mark D. Noar, Owings Mills, MD (US)

(72) Inventor: Mark D. Noar, Owings Mills, MD (US)

(21) Appl. No.: 16/881,052

(22) Filed: May 22, 2020

Related U.S. Application Data
(60) Provisional application No. 62/857,297, filed on Jun. 5, 2019.

(51) Int. Cl. G01N 33/68

(52) U.S. Cl. CPC G01N 33/6893; G01N 2800/60; G01N 2333/71; G01N 2333/7155

(57) ABSTRACT

A method identifies an adhesion in a human subject by obtaining a sample of a bodily fluid derived from the subject, and assaying the sample for activity of a biomarker specific for an adhesion in internal tissue of the subject to thereby determine whether the subject has an internal adhesion. Another method identifies an adhesion in a human subject by administering an imaging agent to the subject with the imaging agent identifying a biomarker specific for an adhesion in internal tissue of the subject, and utilizing imaging equipment to locate the adhesion identified by the imaging agent.

### BACKGROUND

Adhesions or scar tissue in humans typically results from abdominal and pelvic surgeries or acute traumatic injury, or infections and can develop between any abdominal surfaces, structures or organs during the healing process. Adhesions can cause intestinal, fallopian tube, or bowel obstruction, chronic pain, infertility or can cause complications when further surgery is required. There are no specific laboratory tests associated with adhesion disease. Current testing for adhesions in the abdomen or pelvic area utilize costly imaging procedures such as MRI. Laparoscopy can also diagnose and perhaps provide treatment of adhesions, but such surgery may be unnecessary if the laparoscopy finds that subject does not have adhesions.

Thus, there is a need to provide a cost-effective diagnostic test determine if adhesions are present in a patient by identifying at least one adhesion marker in circulating bodily cells, body fluids, or plasma, and to permit treatment of the adhesions without surgery.

### SUMMARY

An objective of the embodiment is to fulfill the need referred to above. In accordance with the principles of an embodiment, this objective is achieved by providing a method that identifies an adhesion in a human subject by obtaining a sample of a bodily fluid derived from the subject, and assaying the sample for activity of a biomarker specific for an adhesion in internal tissue of the subject to thereby determine whether the subject has an internal adhesion.

In accordance with another aspect of an embodiment, a method identifies an adhesion in a human subject by administering an imaging agent to the subject with the imaging agent identifying a biomarker specific for an adhesion in internal tissue of the subject, and utilizing imaging equipment to locate the adhesion identified by the imaging agent.

Other objectives, features and characteristics of the present embodiment, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawing.

FIG. 1 is a flowchart of method steps of an embodiment.

FIG. 2 is a flowchart of method steps of another embodiment.

### DETAILED DESCRIPTION OF EXAMPLE EMBODIMENTS

After abdominal or pelvic surgery, internal scar tissue (adhesions) may form between two tissue surfaces during healing. The scar tissue forms since the rejoining tissue does not grow the collagen back the same way it was originally, or the scar tissue forms as a result of disordered reaction of myofibroblasts due to tissue manipulation. Thus, scar tissue is a disordered, benign tissue growth process than can cause pain, create an obstruction, and cause complications when further surgery is required. This disclosure provides methods for identifying internal adhesions and treating or reducing such adhesions without the need for surgery.

Fibroblast cells transition to myofibroblasts cells during scaring in an acute development phase of scaring. Biomarkers in the myofibroblasts cells can be identified in the subject's bodily fluid such as blood, plasma and/or at the site of the adhesion. The biomarkers can include circulating cells, proteins or end products as result of transition. The proteins are preferably chemokines or cytokines (TGF-β1, IL-6 and IL-8). Other indicators of adhesions can include the expression of α-SNA stress fibers during the transition of fibroblast cells to myofibroblast cells, excessive collagen and fibonectin secretion, expression of fibrotic ECM proteins such as SPARC and Tenascin, low MMP secretion, and immune cells such as mast cells, monocytes, T cells, etc.). Also, the involvement of the TGF-β/SMAD and Wnt/β-catenin pathways are known to be important in developing scars and can be considered biomarkers.

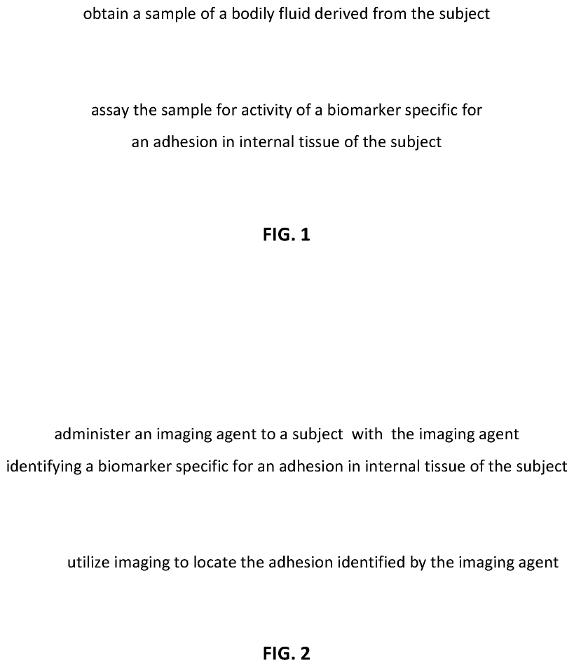

In accordance with an embodiment and with reference to FIG. 1, a method of identifying an adhesion in a subject includes, in step 10, obtaining a sample of a bodily fluid derived from the subject under test. In step 20, the sample of a bodily fluid is assayed for activity of a biomarker specific for an adhesion in internal tissue of the subject. Thus, when the biomarker is present in sufficient quantity, it is determined that the subject has an internal adhesion.

In accordance with another aspect of an embodiment and with reference to FIG. 2, a method of identifying an adhesion in a subject includes in step 30, administering an imaging agent to a subject. The imaging agent is configured to identify a biomarker specific for an adhesion in internal tissue of the subject. In step 40, imaging equipment is used to locate the adhesion identified by the imaging agent. The imaging agent can be any substance that can identify the biomarker and be seen on imaging equipment, such as a contrast agent, a radioactive contrast agent or other similar agents. The imaging agent can be administered by mouth, enema, or injection into a vein, artery, or body cavity. The biomarker is preferably the proteins or the other indicators as noted above.

The myofibroblast cells are intermediate cells and may not be available to identify a chronic adhesion in the subject. Chronic, unhealthy wounds or adhesions have been found to include biomarkers such as the proteins IL1, IL6, and matrix metalloproteinases (MMPs). Thus, the above methods of identifying an adhesion can be employed to identify chronic adhesions by identifying these or other biomarkers indicative of a chronic adhesion.

Once an adhesion or adhesions are identified by the methods disclosed, this information can be used by a physician to determine if surgery or other treatment would be beneficial to alleviate pain or other issues caused by the adhesions. To treat the identified internal adhesions without surgery, the methods disclosed can further include targeting the internal adhesion that provides the biomarker by injecting into a vein, artery, or body cavity of the subject, chemicals, such as steroids, antibodies or other substances that can destroy or reduce the cells defining the adhesion and/or prevent collagen formation at the adhesion site. In addition, knowing that subject has adhesions can be valuable pre-operative information for a surgeon performing open or laparoscopic surgery near the adhesions.

Thus, the methods disclosed advantageously provide a way to determine if a human subject has adhesions by monitoring biomarkers and thus, does not require surgery to determine and locate internal bodily adhesions. Furthermore, non-surgical reduction treatment of internal adhesions is also possible.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A method of identifying an adhesion in a human subject, the method comprising the step of:
obtaining a sample of a bodily fluid derived from the subject; and
assaying the sample for activity of a biomarker specific for an adhesion in internal tissue of the subject to thereby determine whether the subject has an internal adhesion.

2. The method of claim 1, further comprising:
after an internal adhesion is determined, targeting the internal adhesion by providing to the subject, chemicals that can destroy or reduce cells defining the internal adhesion and/or prevent collagen formation at a site of the internal adhesion.

3. The method of claim 1, wherein the biomarker comprises cells.

4. The method of claim 3, wherein the cells are myofibroblasts cells.

5. The method of claim 3, wherein the cells are mast cells, monocytes, or T cells.

6. The method of claim 1, wherein the biomarker comprises at least one protein.

7. The method of claim 1, wherein the biomarker comprises cytokine proteins.

8. The method of claim 6, wherein the cytokine proteins include TGF-β1, IL-6 and IL-8.

9. The method of claim 5, wherein the adhesion is a chronic adhesion and the at least one protein incudes protein IL1 or protein IL6.

10. The method of claim 3, wherein the bodily fluid is blood and the cells are circulating cells.

11. A method of identifying an internal adhesion in a human subject, the method comprising the steps of:
administering an imaging agent to the subject, the imaging agent identifying a biomarker specific for an adhesion in internal tissue of the subject, and
utilizing imaging equipment to locate the internal adhesion identified by the imaging agent.

12. The method of claim 11, further comprising:
after an internal adhesion is located, targeting the internal adhesion by providing to the subject, chemicals that can destroy or reduce cells defining the internal adhesion and/or prevent collagen formation at a site of the internal adhesion.

13. The method of claim 11, wherein the biomarker comprises cells.

14. The method of claim 13, wherein the cells are myofibroblasts cells.

15. The method of claim 13, wherein the cells are mast cells, monocytes, or T cells.

16. The method of claim 11, wherein the biomarker comprises at least one protein.

17. The method of claim 11, wherein the biomarker comprises cytokine proteins.

18. The method of claim 17, wherein the cytokine proteins include TGF-β1, IL-6 and IL-8.

19. The method of claim 16, wherein the adhesion is a chronic adhesion and the at least one protein incudes protein IL1 or protein IL6.

20. The method of claim 11, wherein imaging agent is a contrast agent or a radioactive contrast agent.
